(19) United States
(12) Patent Application Publication
(10) Pub. No.: US 2024/0358344 A1
(43) Pub. Date: Oct. 31, 2024

(54) SLIP RING SYSTEM FOR TRANSFERRING ENERGY INTO A ROTATING INSTALLATION

(71) Applicant: Siemens Healthineers AG, Forchheim (DE)

(72) Inventors: Hans-Juergen MUELLER, Pretzfeld (DE); Viktor LOEWEN, Sankt Augustin (DE)

(73) Assignee: Siemens Healthineers AG, Forchheim (DE)

(21) Appl. No.: 18/645,451

(22) Filed: Apr. 25, 2024

(30) Foreign Application Priority Data
Apr. 27, 2023 (DE) 10 2023 203 898.6

(51) Int. Cl. A61B 6/00; A61B 6/03

(52) U.S. Cl. CPC A61B 6/56; A61B 6/035; A61B 6/4441

(57) ABSTRACT

A slip ring system comprises a slip ring component including a number of sliding contact tracks, the sliding contact tracks being arranged coaxially around an axis of rotation, the slip ring component being C-shaped and forming a curved shape with an opening with an opening angle greater than 20° with respect to the axis of rotation; and a contact component including a plurality of brush elements which are arranged at different positions in accordance with a rotation around an arrangement angle around the axis of rotation in such a manner that they can make electrically conductive contact therewith, the arrangement angle of the brush elements relative to each other is greater than the opening angle of the opening.

### CROSS-REFERENCE TO RELATED APPLICATION(S)

The present application claims priority under 35 U.S.C. § 119 to German Patent Application No. 10 2023 203 898.6, filed Apr. 27, 2023, the entire contents of which is incorporated herein by reference.

### FIELD

One or more example embodiments of the present invention relates to a slip ring system for transferring energy into a rotating installation. Furthermore, one or more example embodiments of the present invention relates to an installation with such a slip ring system and a method for supplying energy in such an installation. In particular, one or more example embodiments of the present invention relates to a slip ring for transferring energy for a CT system with open C-geometry (and complete 360° rotation).

### RELATED ART

Slip ring systems have been known in the prior art for a long time. In this case, these involve an assembly consisting of a slip ring with annular contact tracks and a so-called brush which makes sliding contact with these contact tracks. Slip ring systems enable the transfer of electrical currents and signals between components which rotate with respect to one another.

In particular, slip rings are used in the field of computed tomography (CT). CT systems or their scanner (also referred to as “gantry”) rotate around the patient (360°) in order to generate a complete CT image.

In the case of intervention CT or CT on operating tables, it is of advantage if the gantry can be moved laterally away the patient. Instead of a closed circle of a conventional CT system, a laterally open circle or at least a gantry that can be opened laterally must be used.

### SUMMARY

CT systems that can be moved laterally away from the patient (table) are known in the prior art. For this purpose, the closed ring structure of the scanner with all its functions, i.e. storage, drive, data, current transfer, cladding, etc., is opened, the scanner is moved and the structure is then closed again. This is disadvantageous since it requires complicated, heavy and expensive technical solutions to implement this function.

It is the object of the present invention to provide a slip ring system for transferring energy into a rotating installation, the slip ring system being designed in particular for a computed tomography system (CT system). In particular, one or more example embodiments of the present invention transfers energy via an open slip ring in a contact-encumbered manner to a rotating system, in particular a CT scanner.

This is achieved by a slip ring system in accordance with claim 1, an installation with such a slip ring system in accordance with claim 9 and a method for supplying energy in such an installation in accordance with claim 11.

### BRIEF DESCRIPTION OF THE DRAWINGS

The invention will be explained again in more detail below with reference to the attached figures using examples of embodiments. In the various figures, identical components are provided with identical reference numerals. The figures are generally not to scale. In the drawings:

FIG. 1 shows an exemplary embodiment of a computed tomography system in accordance with the prior art,

FIG. 2 shows a perspective view of an example of a slip ring system in accordance with one or more example embodiments of the present invention with sliding contact tracks on the end face of the slip ring component,

FIG. 3 shows a perspective view of an example of a slip ring system in accordance with one or more example embodiments of the present invention with sliding contact tracks on the outer surface of the slip ring component,

FIG. 4 shows a mobile CT system comprising a gantry with a slip ring system in accordance with one or more example embodiments of the present invention in multiple rotation states,

FIG. 5 shows a preferred brush unit for four sliding contact tracks,

FIG. 6 shows an outline of a brush element in electrical contact with a slip ring component,

FIG. 7 shows alternative for a preferred separation of an electrical contact between the slip ring component and brushes with eccentric rollers,

FIG. 8 shows an alternative for a preferred separation of an electrical contact between the slip ring component and brushes with a lift unit, wherein current is switched off after the separation,

FIG. 9 shows an alternative for a preferred separation of an electrical contact between the slip ring component and brushes with inclined ends of the slip ring component, wherein current is switched off shortly before loss of contact,

FIG. 10 shows an alternative for a preferred separation of an electrical contact between the slip ring component and brushes with a hinge, wherein current is disconnected after a different brush unit has been connected,

FIG. 11 shows a brush element with eccentric rollers,

FIG. 12 shows its brush element with a hinge,

FIG. 13 shows an embodiment for a passive separation of the brush element and slip ring component, and

FIG. 14 shows the sequence for a passive separation

of the brush element and slip ring component.

### DETAILED DESCRIPTION

A slip ring system in accordance with one or more example embodiments of the present invention is used, for example, to transfer energy into a rotating installation, for example a CT scanner. The slip ring system comprises a slip ring component with a number of sliding contact tracks, which are arranged coaxially around an axis of rotation, and a contact component, wherein the components are arranged rotatably relative to one another around the axis of rotation and are designed such that the number of sliding contact tracks make electrically conductive contact with the contact component via a number of brushes (which slide over the sliding contact tracks) during rotation.

The slip ring system is characterized in that the slip ring component is C-shaped and forms a curved shape with an opening with an opening angle greater than 20°, and the contact component comprises a plurality of brush elements which are arranged at different positions on the slip ring component in accordance with a rotation around an arrangement angle around the axis of rotation and make electrically conductive contact therewith, and the arrangement angle of the brush elements relative to each other is greater than the opening angle of the opening.

As already mentioned above, it is known that a slip ring system has a slip ring (the “slip ring component”) with at least one sliding contact track. Generally, there are multiple sliding contact tracks, since current must always flow, i.e. at least one sliding contact track for a positive pole and one sliding contact track for a negative pole. The sliding contact tracks are located on the surface of the slip ring and are electrical insulated from one another, consequently there is no short circuit between the sliding contact tracks. For example, the sliding contact tracks can be copper tracks and/or metal curves on the surface of the slip ring.

So that current can be transferred, the slip ring system also comprises a contact component with brushes which enable electrical contact with the sliding contact tracks. The sliding contact tracks are arranged coaxially with one another, so that during a rotation of the slip ring around a point of arrangement) there is rotation (the center of the coaxial continuous contact with the brushes.

The special feature of the slip ring system in accordance with one or more example embodiments of the present invention is that the slip ring (and thus also its sliding contact tracks) is C-shaped, i.e. open. This opening is also comparatively large with an opening angle greater than 20°, i.e. a center point angle of the curved shape less than 340°. The opening can be configured in particular in a radial manner. In particular, the arrangement angle can be an angular distance in relation to the axis of rotation.

Due to the size of the opening (for a slip ring with a diameter of one meter, the opening would be larger than 17 cm), continuous contact transfer using one brush per sliding contact track is problematic. The contact component therefore comprises a plurality of brush elements (with at least one brush per sliding contact track), which are arranged at different positions on the slip ring component and form an electrically conductive contact therewith.

Looking at the arrangement of these brush elements, it is required that they are arranged around the axis of rotation (of the slip ring system) in accordance with a rotation around an arrangement angle. The arrangement angle is fixed and corresponds to an angle of rotation by which the brush elements are rotated relative to each other around the axis of rotation. In this case, the arrangement angle must be greater than the opening angle of the opening so that at least one brush element is always in electrical contact with its sliding contact tracks in every position of the slip ring (i.e. the slip ring component).

An installation in accordance with one or more example embodiments of the present invention with a rotatable component comprises a slip ring system in accordance with one or more example embodiments of the present invention. It is preferred that the installation is a computed tomography system and comprises a gantry (i.e. a CT scanner) with a stator and a rotator as rotatable components. The slip ring system transfers energy and signals between the stator and rotator. In this case, the gantry is C-shaped and has a lateral opening (i.e. in the side of the ring), through which an object to be examined, in particular a human, can be moved laterally into the gantry. In this case, the opening of the slip ring component is preferably greater than or equal to this opening. In this case, it is preferred that the slip ring component of the slip ring system is attached to the rotator and the contact component of the slip ring system is attached to the stator, however a precisely reverse arrangement is also possible in which the slip ring component of the slip ring system is attached to the stator and the contact component of the slip ring system is attached to the rotator.

In the case of a method in accordance with one or more example embodiments of the present invention for supplying energy in an installation in accordance with one or more example embodiments of the present invention, an electrical contact for a current flow between an energy supply (on the stator) and a load in the rotatable component of the installation (rotator) is established via the slip ring system in accordance with one or more example embodiments of the present invention and the rotatable component is rotated.

Further, particularly advantageous embodiments and developments of the invention are disclosed in the dependent claims and the description below, wherein the claims of one claim category can also be developed in a similar manner to the claims and description parts relating to another claim category and in particular also individual features of different exemplary embodiments or variants can be combined to form new exemplary embodiments or variants.

In accordance with a preferred slip ring system, the slip ring component has a C-shaped curved shape with an end face and an outer surface. It therefore has the shape of a (thin) cylinder with an opening on the side. The end faces have the shape of a circular ring curve, the outer surfaces have the shape of a curved strip with an interruption where the opening is. A number (preferably a plurality) of sliding contact tracks are arranged on the outer surface of the slip ring component. These sliding contact tracks are concentric but spaced from one another with respect to the height of the outer surface (thickness of the ring). Alternatively or additionally, a plurality of sliding contact tracks is arranged concentrically to one another with different radii on the end face of the slip ring component.

In this case, it is particularly preferred that each sliding contact track is assigned in each case at least one brush of a plurality of brush elements. This means that one brush per sliding contact track can lose contact with the slip ring component due to the opening in the slip ring component, but the other brush still makes electrical contact with this sliding contact track.

According to a preferred slip ring system, the opening angle is less than 180°, the slip ring component is therefore at least a semicircle. The contact component comprises in this case at least one pair of brush elements, but three or more brush elements may also be present in the case of large openings.

The arrangement angle of the two brush elements to each other should preferably be between 120° and 240°, particularly preferably between 160° and 200°. Care should always be taken to ensure that at least one brush element always makes electrical contact with the sliding contact tracks in every orientation of the slip ring component. In practice, an opening is preferred that is just large enough to allow an intended object to be inserted laterally through the opening of the slip ring component. In the case of a C-shaped CT scanner (the “gantry”), it is advantageous in practice if the opening angle is less than or equal to 120°, i.e. the slip ring corresponds to at least a two-thirds circular ring. In this case, an opposing arrangement of the brush elements (arrangement angle 180°) can represent a simple design that always ensures electrical contact. Three brush elements with a respective arrangement angle of 120° (in an equilateral triangle) could also be used. This could compensate for the failure of a brush component.

When the slip ring component rotates, individual brush elements of the contact component repeatedly lose contact with their sliding contact tracks due to the opening of the slip ring component. This could be accompanied by a disturbance of the current flow, for example due to sparking and lead to fluctuations in the energy supply or interference pulses during signal transfer. Particularly when recording measurement data, for example in the case of CT scanners, interference should therefore be suppressed if a brush element loses contact.

Since the brushes of the brush elements are usually spring-mounted or formed from springs, mechanical stresses could also occur when a contact is re-established by an edge of the slip ring component. This could lead to a reduction in the service life of brushes but also to further disturbances in the current flow.

There are multiple options to overcome these disadvantages. In all of these options, a loss of contact is specifically initiated and re-established. This can be performed, for example, via inlet and outlet inclines in the slip ring component or by lifting the brush element, or the electrical contact of the relevant brush element with a current supply or measuring station can be switched off immediately before lifting or positioning, so that no voltage peaks or damage to the brush or the sliding contact track occur.

A preferred slip ring system is shaped and/or designed so that when a brush element is rotated across an end region of the slip ring component facing the opening, an electrically conductive contact is established between the slip ring component and a brush element on the slip ring component outside the end region. The “end region” should be at least 5 mm, in particular at least 1 cm or even at least 5 cm (up to the opening, i.e. the end of the slip ring component in the direction of rotation), so that the contact of a brush can still be established or separated via a sliding contact track even in the event of rapid rotation.

Preferably, the slip ring system is shaped and/or designed in such a manner that, in the case of a relative movement of a brush element directed toward the opening, the electrically conductive contact first separates and only then is the end region passed and/or that, in the case of a relative movement of a brush element directed from the opening toward the slip ring component, the end region is first passed and only then is the electrically conductive contact established. It is particularly preferred that the electrical contact is released before reaching the opening and only re-established after the end region has been passed over again.

Particularly preferably, the slip ring system is shaped or designed in such a manner that, in the case of a relative movement of a brush element directed toward the opening in a transition region of the slip ring component adjacent to the end region, the relative distance between the slip ring component and a holder of the brushes of the contact component increases continuously so that the electrically conductive contact separates before the end region is reached. The brush element therefore moves away from the surface of the slip ring component with a relative movement until the contact separates. Conversely, it is correspondingly preferred that, in the case of a relative movement of a brush element directed away from the opening in a transition region of the slip ring component adjacent to the end region, the relative distance between the slip ring component and a holder of the brushes of the contact component decreases continuously so that the electrically conductive contact is established after crossing the end region. This can be achieved, for example, by lifting brushes or by an end incline of the slip ring component.

In accordance with a preferred slip ring system, the slip ring component is shaped in the end region, and preferably also in a transition region, in such a manner that it constantly distances itself from a rotationally symmetrical outer surface in which the contact elements are located. This outer surface would correspond to a circular cylindrical outer surface and “distances itself” means that the surface tapers inward in a wedge shape. These are the aforementioned end inclines.

In this case, it is preferred that a number of the sliding contact tracks are arranged on the end face of the slip ring component and that the end region, and preferably also the transition region, is shaped in such a manner that the end face with the number of sliding contact tracks there distances itself from a rotationally symmetrical plane on which the remaining end face lies (i.e. a ring with wedge-shaped ends with respect to the end face).

Alternatively or additionally, it is preferred that a number of the sliding contact tracks are arranged on the outer side of the slip ring component and that the end region, and preferably also the transition region, is shaped in such a manner that the end face with the number of sliding contact tracks there distances itself from a rotationally symmetrical outer surface on which the remaining end face lies (i.e. a ring with wedge-shaped ends with respect to the outer surface).

Since the brush elements are usually spring-mounted, the spring travel of the brushes in the embodiment described above should be designed in such a manner that the brushes only hit the “run-in incline” above the slip ring component (i.e. in the transition region) (without being damaged at the edge) or contact is broken even before the edge is reached.

According to a preferred slip ring system, the contact component has movement elements that are designed so as to move brushes of brush elements relative to the slip ring component so that the distance between the brushes and the slip ring component and/or the pressure of the brushes on the slip ring component can be changed. This movement can be active or passive. For active movement, the slip ring system preferably comprises a control apparatus that is designed so as to measure the position of a brush element relative to the slip ring component and, in the event that a brush element approaches one end of the slip ring, to actively control the movement element (i.e. to lift it off when it leaves the slip ring component and to put it back on when it has reached the slip ring component again).

A movement element preferably comprises a hinge and a tilting apparatus via which a brush element can be tilted relative to the surface of the slip ring component. In this case, a motorized, pneumatic, magnetic or hydraulic pivoting of the entire brush element is preferably performed so that the brushes of the entire brush element are moved further away from or closer to the sliding contact tracks.

A movement element preferably comprises a brush lifting apparatus via which at least one brush of a brush element can be moved closer to the slip ring component or further away from the slip ring component, preferably wherein the brush lifting apparatus acts on a brush holder or on individual brushes. A preferred brush lifting apparatus is an eccentric guide in which an axis is arranged eccentrically parallel to a motor axis and acts orthogonally on springs of the brushes, so that, when the motor axis is rotated, the brushes are moved further away from or closer to the sliding contact tracks.

A movement element preferably comprises a lifting apparatus via which a brush element can be moved closer to the slip ring component or further away from the slip ring component. The lifting apparatus preferably acts on a complete brush element, for example on a mounting block of the brushes. Via a motorized, pneumatically, magnetically or hydraulically induced movement, the entire brush element can then be moved further away from the sliding contact tracks or closer to them.

The spring travel of the brushes should of course be taken into account for the respective movements.

According to a preferred slip ring system, the slip ring component has a number of slide rails in the end region and said slid rails preferably also in the transition region, protrude in a curved shape (along the direction of rotation) from the surface of the slip ring component and in particular extend up to the opening. The brush elements have sliding elements which are shaped and arranged in such a manner that, in the case of a relative movement of the slip ring component and brush unit, they slide on the slide rails and guide the brush unit along the curved shape of the slide rail. Due to the curved shape of the slide rails, the brushes are constantly lifted off the surface of the sliding contact tracks or moved toward them as they slide over them. The curved shape is preferably mirror-symmetrical, so that a brush element that hits one end of the slip ring component from the opening is first lifted on the slide rail by the sliding element and then gently placed down on the sliding contact track, accordingly.

As mentioned above, it is also possible, in addition to lifting off or placing down the brushes on the sliding contact tracks, to interrupt the electrical contact via a switch. This can be performed alternatively but in particular preferably in a combination.

A preferred slip ring system comprises a control apparatus which is designed so as to control the current flow through the slip ring system in such a manner that before an electrically conductive contact between a brush element and the slip ring component separates, the current flow through this contact is interrupted and after an electrically conductive contact between a brush element and the slip ring component is established, an interrupted current flow through this contact is re-established. For this purpose, the control apparatus preferably comprises an electronic or mechanical position detection system that measures whether a brush element has reached the transition region or the end region and then switches off the electrical contact of the brush element via a switch. The contact with a load or measuring unit in the rotating part can be switched off and/or with an energy supply or a data acquisition unit in the static part.

A preferred position detection system measures electronically or simply mechanically. “Position detection” also means that a switch on the slip ring component is operated by a lever on the brush unit or conversely. The electrical contact between the brush element and the slip ring component is therefore not disconnected here, but rather the electrical contact between the brush element and components outside the slip ring system, wherein, as already mentioned, a combination with the aforementioned embodiments is particularly preferred, since this prevents both electrical interference and mechanical damage to the brushes.

Preferably, the control apparatus is designed so as to interrupt the contact of the brush element with an energy supply or to interrupt the contact of the brush element to a load. In an embodiment in which the brush element is arranged on the rotator, its contact with the load or with a sensor or detector or a data interface is preferably to be interrupted.

According to a preferred method, in the case of a relative movement of a brush element directed toward the opening of the slip ring component, its brushes are moved away from the slip ring component via a movement element so that their electrically conductive contact with the sliding contact tracks separates, in particular before an end region of the slip ring system is passed.

According to a preferred method, in the case of a relative movement of a brush element directed away from the opening of the slip ring component, its brushes are moved toward the slip ring component via a movement element, so that first the end region is passed and then an electrically conductive contact is established between the brushes and the slip ring component.

According to a preferred method, in the case of a relative movement of a brush element directed toward the opening of the slip ring component, the current flow through this brush element is switched off in particular before it passes an end region of the slip ring system.

According to a preferred method, in the case of a relative movement of a brush element directed away from the opening of the slip ring component, the current flow through this brush element is switched on in particular after it has passed the end region.

The advantage of one or more example embodiments of the present invention is that an open slip ring can be realized. Only then is it possible to realize a simple whole-body CT for interventional use, with which it is possible to move laterally over a patient. This brings many advantages for the doctor and patients during examinations, especially during surgical interventions on patients. In particular, time savings, better accessibility and, last but not least, cost-effective implementation are worth mentioning. The closed slip rings previously used in CT systems are highly reliable. This is also achieved with the open slip ring system in accordance with one or more example embodiments of the present invention. A closing and opening mechanism in a CT scanner is no longer necessary, which allows a simple mechanical solution with high reliability. Since part (in the practical implementation approx. one third) of the slip ring is not present, there is also a weight saving, which is particularly advantageous during transportation and for the floor loading of mobile CT systems.

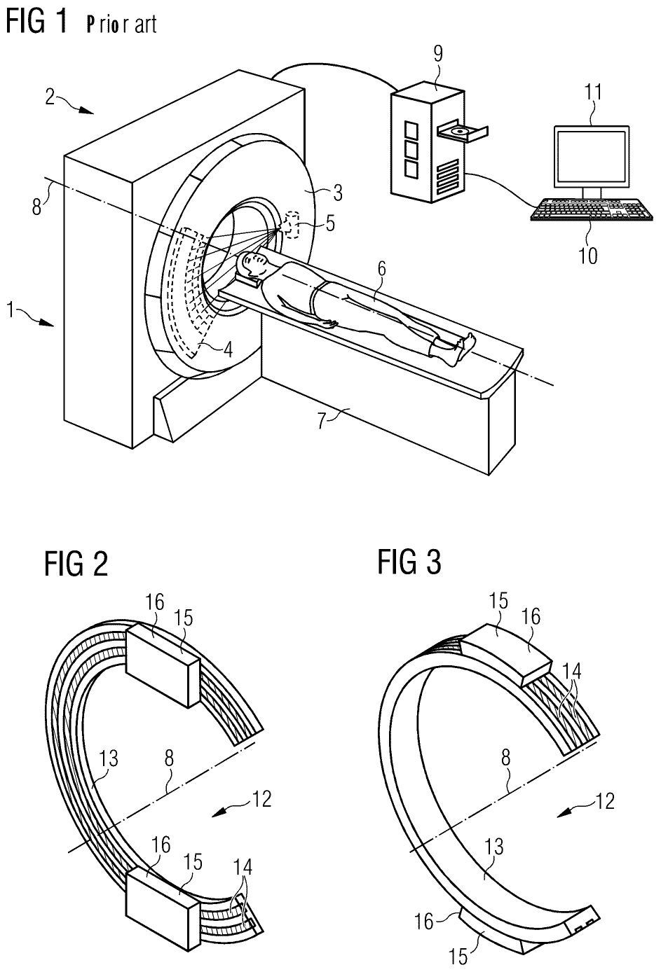

FIG. 1 shows an embodiment of a computed tomography system (CT system) 1 as an example of a device system 1 with a radiation detector 4 and a radiation source 5. The radiation source 5 is designed so as to illuminate the radiation detector 4 with radiation. The illustrated CT system 1 comprises a gantry 2 with a rotor 3. The rotor 3 comprises as a radiation source 5 an X-ray source and the radiation detector 4 which is designed so as to detect X-ray radiation.

The rotor 3 can rotate around the axis of rotation 8. The examination object 6, in this case a patient, is positioned on the patient couch 7 and can be moved along the axis of rotation 8 through the gantry 2. The computing unit 9 is provided in order to control the imaging system 1 and/or to generate an X-ray image data set based on signals detected by the radiation detector 4.

In the case of a computed tomography device, a (raw) X-ray image data set of the object 6 is usually recorded from a multiplicity of angle directions via the radiation detector 4. Subsequently, a final X-ray image data set can be reconstructed based on the (raw) X-ray image data set via a mathematical method, for example comprising a filtered back projection or an iterative reconstruction method.

The computing unit 9 can comprise a control unit for controlling the CT system 1 and a generating unit for generating an X-ray image data set.

Furthermore, an input facility 10 and an output facility 11 are connected to the computing unit 9. The input facility 10 and the output facility 11 can enable an interaction by a user or the display of a generated X-ray image data set or output a determined solution to the problem.

FIGS. 2 and 3 show a perspective view of an example of a slip ring system 12 in accordance with one or more example embodiments of the present invention with sliding contact tracks 14 which are arranged concentrically around an axis of rotation 8.

In FIG. 2, the sliding contact tracks 14 are arranged on the end face of the slip ring component 13 or are embedded in its end face. The contact component 15 which is formed from two opposing brush elements 16 is positioned over the end face in such a manner that the brushes 17 (shown, e.g., in FIG. 5) of the brush elements 16 make electrical contact with the sliding contact tracks 14.

In FIG. 3, the sliding contact tracks 14 are arranged on the outer surface of the slip ring component 13 or are embedded in its outer surface. The contact component 15 which is also formed here from two opposing brush element 16 is positioned over the outer surface in such a manner that the brushes 17 of the brush elements (not visible here, see following figures) make electrical contact with the sliding contact tracks 14.

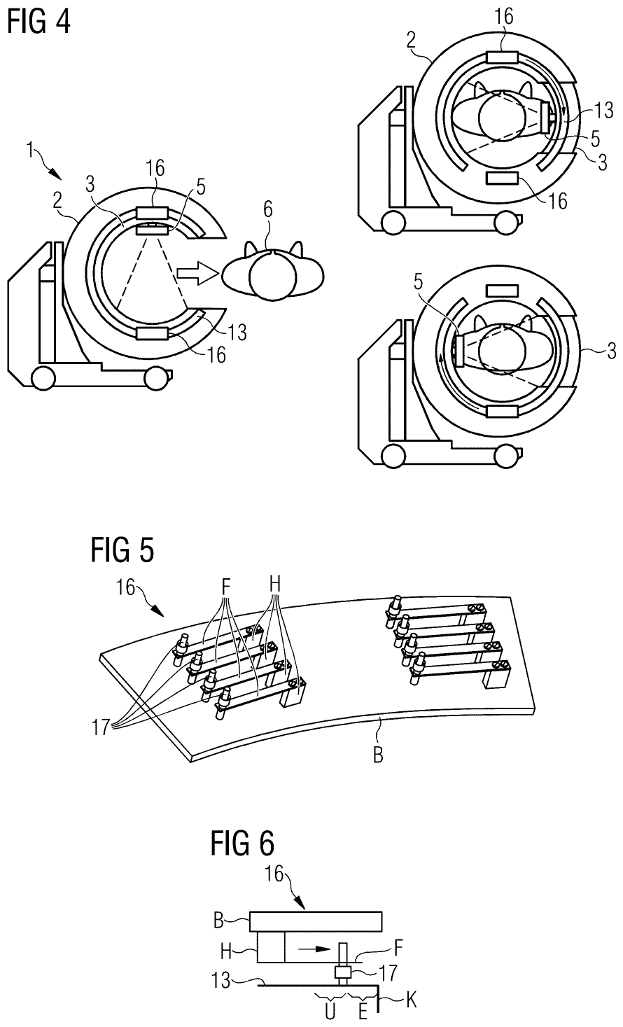

FIG. 4 shows a mobile CT system 1 comprising a gantry 2 with a slip ring system 12 in accordance with one or more example embodiments of the present invention in multiple rotation states. The view is of the end face of the gantry 2 or of the slip ring system 12.

The CT system 1 can be seen in the open state on the left. The radiation source 5 is arranged at the top and the radiation detector 4 (not visible here) is arranged at the bottom together with the slip ring component 13 in the rotator 3. The brush units 16 are arranged on the stator, i.e. on the housing of the gantry 2. The CT system is pushed for an examination in the direction of the arrow over a patient as an examination object 6, who could rest, for example, on a couch (not illustrated).

Two different phases of the examination are illustrated on the right. The rotator 3 now rotates in the direction of the arrow around the patient while the radiation source 5 irradiates the radiation detector 4. It can be clearly seen that in the top image the lower brush unit 16 is not in contact with the slip ring component 13, since it is located precisely in its opening, and in the lower image the upper brush unit 16. However, this is irrelevant, since at least one brush unit 16 is always in contact with the slip ring component 13 and energy and signals can be transferred via it.

FIG. 5 shows a perspective view of a preferred brush unit 16 for four sliding contact tracks 14, which are arranged on the end face of a slip ring component (see for example FIG. 2). There is a pair of brushes 17 for each of the four sliding contact tracks 14, which serves to ensure transmission reliability. Each brush is elastically connected to a common fastening element B via a spring steel F on a holder H. The brush unit 16 can be attached to a stator or a rotator 3 via this fastening element B.

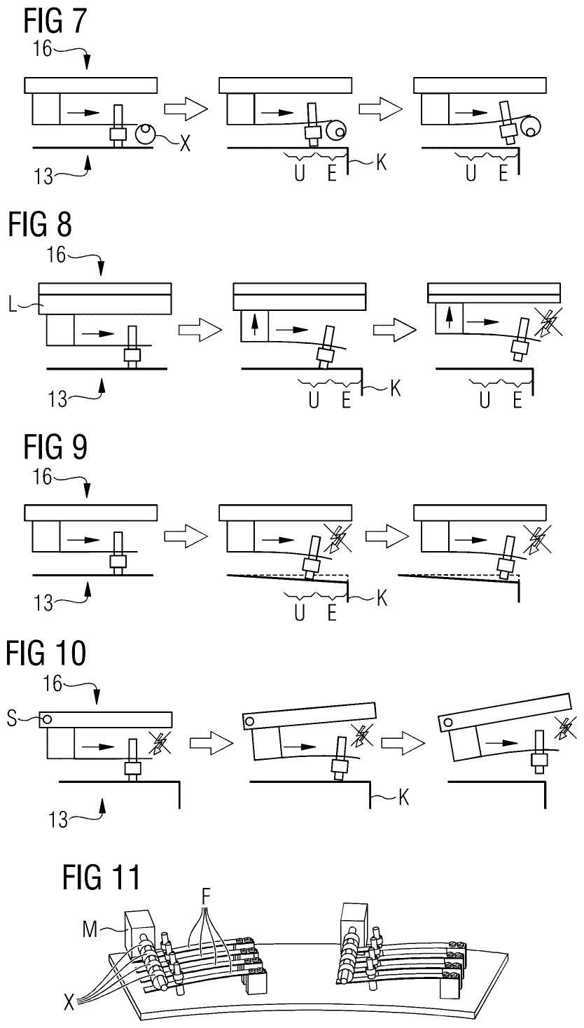

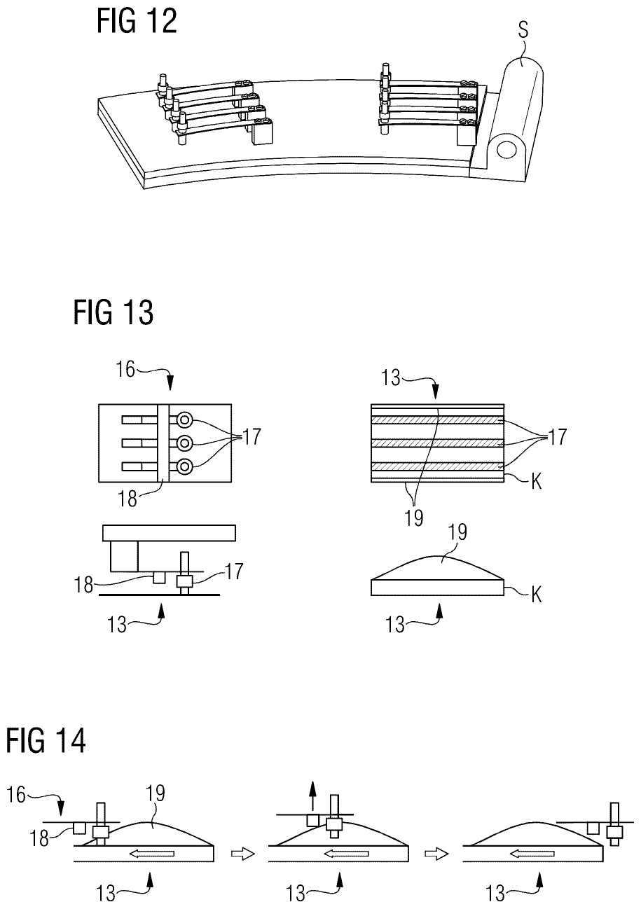

FIG. 6 shows an outline of a brush element 16 in electrical contact with a slip ring component 13. In this figure, as in the following FIGS. 7 to 10, it should be noted in comparison with FIG. 5 (and the following FIGS. 11 and 12) that the contact surface of the brush 17 points downward and that the force of gravity should also act downward (hence sometimes a deflection of the spring steel F). FIGS. 5, 11 and 12 lie on their backs, so to speak, so that the contact surfaces of the brushes 17 point upward.

The brush element here comprises brushes 17 which are arranged on a fastening element B via spring steel F and holder H, as in FIG. 5. The brush 17 slides here in the direction of the arrow straight through the transition region U toward its edge K, where the opening begins.

It would be desirable if the brush 17 were to lose electrical contact with the slip ring component 13 before the end region E. The following figures show how this can be achieved.

FIGS. 7 to 10 show various alternatives as to how electrical contact between the slip ring component 13 and the brush could be separated. The preferred method of establishing contact is the reverse of separation. All these alternatives could also be combined with each other. Shown from left to right is a series of three illustrations in which a brush 17 moves toward an opening in a slip ring component 13 and separates from the slip ring component 13 shortly before reaching an end region E (in a transition region U).

FIG. 7 shows an alternative for a preferred separation of an electrical contact between the slip ring component 13 and brush 17 with eccentric rollers X. These are arranged on an axis of a motor M (not shown here, see FIG. 11) and designed in such a manner that they can raise or lower the brush 17 when the motor M rotates.

FIG. 8 shows an alternative for a preferred separation of an electrical contact between the slip ring component 13 and brush 17 with a lift unit L. The closer the brush 17 comes to the edge K of the slip ring component 13, the more it is lifted by the lift unit L until it separates from the slip ring component 13. In this example, a current flowing through the contact is switched off after separation, which is symbolized by a crossed-out lightning bolt.

It should be noted here for all these examples that the switching-off procedure of one example can also be used for the other examples. In addition, current does not necessarily have to be switched off, but a signal flow can also be interrupted. In general, the electrical contact with an energy supply, with a load, with a sensor, with a data acquisition unit or with a data interface can be interrupted, for example via a switch.

FIG. 9 shows an alternative for a preferred separation of an electrical contact between the slip ring component 13 and brush 17 with inclined ends of the slip ring component 13. This is inclined at its ends in such a wedge shape that the brush 17 loses contact despite the spring force. In this example, the current is switched off shortly before contact is lost, which has the advantage that effects such as arcing, which could occur when contact is lost, are prevented.

FIG. 10 shows an alternative for a preferred separation of an electrical contact between the slip ring component 13 and brush 17 with a hinge S. Here, the brush 17 is not lifted as shown in FIG. 8, but tilted so that it loses contact with the slip ring component 13. In this example, current is disconnected after another brush unit has been connected. It is sufficient if a single brush element 16 is electrically connected to the rotator 3. The brush element 16 that will next lose contact with the slip ring component 13 can therefore easily be disconnected from other components of the installation at any time when a brush element that has just regained contact with the slip ring component 13 has been “switched on”.

FIG. 11 shows a brush element 16 with eccentric rollers X, as already roughly outlined in FIG. 7. The basis is the brush element 16 of FIG. 5. This illustration shows one motor M per brush group, on the axis of which eccentric rollers X are mounted and the individual brushes 17 can be raised and lowered simultaneously on their spring steels F.

FIG. 12 shows a brush element 16 with hinge S (see also FIG. 10), via which the entire brush element 16 can be tilted and thus the contact between brushes 17 and slip ring component 13 can be separated or established.

FIG. 13 shows an embodiment for a passive separation of brush element 16 and slip ring component 13. On the left, a brush element 16 is shown (in the upper illustration from below and in the lower illustration from the side) as it was already shown in FIG. 6, but here it is additionally equipped with a sliding unit 18, with which the brushes 17 and also the entire brush element can be moved.

The slip ring component 13 is shown on the right, which has a slide rail 19 on each of its sides, which are aligned parallel to the relative movement between slip ring component 13 and brush unit 16 and protrude in a curved-shaped manner from the surface of the slip ring component 13. The slide rails 19 are shown above in a plan view and below from the side.

FIG. 14 shows the sequence of a passive separation of brush element 16 and slip ring component 13 with a variant according to FIG. 13. The sliding element 18 is shaped and arranged in such a manner that, in the case of a relative movement of the slip ring component 13 and the brush unit 16 along the outlined arrow, it slides on the slide rails 19 and can guide the brush unit 16 along the curved shape of the slide rail 19.

From left to right, which corresponds to a lift-off before reaching the opening, the brush 17 with the sliding element 18 reaches the slide rail 19 (left), is pressed upward by it (center), so that the contact between brush 17 and slip ring component 13 separates, and is then guided downward again (right), wherein the brush 17 is now located in the opening.

Viewed from right to left, this would be an example of restoring a contact, which is performed precisely the other way round. It should be noted that the slide rails 19 could also protrude beyond the edge.

Finally, it should be pointed out once again that the methods described in detail above and the system illustrated are merely exemplary embodiments which can be modified by the person skilled in the art in various ways without departing from the scope of the invention. Furthermore, the use of the indefinite articles “a” or “one” does not exclude the possibility that the relevant features may also be present more than once. Likewise, the terms “unit” and “device” do not exclude that the relevant components consist of multiple interacting part components which can where appropriate also be distributed in a spatial manner. The wording “a number” is to be read as “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements

(for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless defined, terms otherwise all (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible language), markup (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A slip ring system, comprising:
a slip ring component including a number of sliding contact tracks, the number of sliding contact tracks arranged coaxially around an axis of rotation, the slip ring component being C-shaped and forming a curved shape with an opening with an opening angle greater than 20° with respect to the axis of rotation; and
a contact component including a plurality of brush elements which are arranged at different positions in accordance with a rotation around an arrangement angle around the axis of rotation in such a manner that they can make electrically conductive contact therewith, the arrangement angle of the brush elements relative to each other is greater than the opening angle of the opening, wherein
the slip ring component and the contact component are arranged rotatably relative to one another around the axis of rotation, the number of sliding contact tracks being electrically conductive contactable with the contact component via a number of brushes of the brush elements.

2. The slip ring system of claim 1, wherein at least one of
the slip ring component includes an end face and an outer surface and the number of sliding contact tracks is arranged on the outer surface of the slip ring component, or
a plurality of sliding contact tracks is arranged concentrically to one another with different radii on the end face of the slip ring component.

3. The slip ring system of claim 1, wherein the opening angle is less than 180° and the contact component comprises at least one pair of brush elements, wherein the arrangement angle of the two brush elements relative to each other is between 120° and 240°.

4. The slip ring system of claim 1, when at least one of the brush elements is rotated across an end region of the slip ring component facing the opening, an electrically conductive contact is established between the slip ring component and the brush element on the slip ring component outside the end region.

5. The slip ring system of claim 4, wherein the slip ring component is shaped in the end region such that the slip ring component is constantly distanced from a rotationally symmetrical outer surface in which contact elements are located.

6. The slip ring system of claim 4, wherein the contact component includes movement elements configured to move brushes of the brush elements relative to the slip ring component such that at least one of a distance between the brushes and the slip ring component or a pressure of the brushes on the slip ring component can be changed.

7. The slip ring system of claim 4, wherein the slip ring component includes a number of slide rails in the end region which protrude in a curve-shaped manner out of a surface of the slip ring component and the brush elements include sliding elements shaped and arranged such that, when the slip ring component and one of the brushes move relative to each other, the sliding elements slide on the slide rails and guide the one of the brushes along the curved shape of the slide rail.

8. The slip ring system of claim 1, further comprising:
a control apparatus including,
an electronic or mechanical position detection system configured to control a current flow through the slip ring system such that before an electrically conductive contact between at least one of the brush elements with the slip ring component separates, the current flow through the electrically conductive contact is interrupted and after another electrically conductive contact between a brush element and the slip ring component is established, an interrupted current flow through the another electrically conductive contact is re-established.

9. An installation comprising:
a rotatable component including the slip ring system of claim 1.

10. The installation of claim 9, wherein the installation is a computed tomography system and further comprises:
a gantry including a stator and a rotator, the rotator being at least part of the rotatable component.

11. A method for supplying energy to the installation of claim 9, comprising:
establishing an electrical contact for a current flow between an energy supply and a load in the rotatable component of the installation via the slip ring system and the rotatable component being rotated.

12. The method of claim 11, wherein when at least one brush element moves relatively toward the opening of the slip ring component, brushes associated with the at least one brush element are moved away from the slip ring component via a movement element such that an electrically conductive contact with the sliding contact tracks separates.

13. The method of claim 11, wherein at least one brush element moves relatively toward the opening of the slip ring system, the current flow through the at least one brush element is switched off.

14. The slip ring system of claim 2, wherein each sliding contact track is assigned at least one brush of a plurality of brush elements.

15. The slip ring system of claim 3, wherein the arrangement angle of the two brush elements relative to each other is between 160° and 200°.

16. The slip ring system of claim 4, wherein the slip ring system is shaped such that at least one of
when the at least one brush element moves relatively toward the opening, the electrically conductive contact first separates and only then passes end region passed, or
when the at least one brush element moves relatively from the opening to the slip ring component, the end region is first passed and then the electrically conductive contact is established.

17. The slip ring system of claim 16, wherein the slip ring system is shaped such that, when the at least one brush element moves relatively toward the opening in a transition region of the slip ring component adjacent to the end region, a relative distance between the slip ring component and a holder of the brushes of the contact component increases continuously such that the electrically conductive contact separates before the end region is reached.

18. The slip ring system of claim 5, wherein the slip ring component is further shaped in a transition region such that the slip ring component is constantly distanced from the rotationally symmetrical outer surface in which contact elements are located.

19. The slip ring system of claim 18, wherein at least one of
a number of the sliding contact tracks are arranged on the end face of the slip ring component and the end region such that the end face with the number of sliding contact tracks is distanced from a rotationally symmetrical plane on which the remaining end face lies, or
number of the sliding contact tracks are arranged on the outer side of the slip ring component and the end region such that the end face with the number of sliding contact tracks there is distanced from a rotationally symmetrical outer surface on which the remaining end face lies.

20. The slip ring system of claim 6, wherein at least one of the movement elements comprises:
a hinge and a tilting apparatus via which one of the brush elements can be tilted relative to the surface of the slip ring component;
a brush lifting apparatus via which at least one brush o can be moved closer to the slip ring component or further away from the slip ring component; and
a lifting apparatus via which a brush element can be moved closer to the slip ring component or further away from the slip ring component.
